(19) United States
(12) Patent Application Publication
(10) Pub. No.: US 2021/0298936 A1
(43) Pub. Date: Sep. 30, 2021

(54) Backdrivable, Electrically Powered Orthotic Device

(71) Applicant: Myomo, Inc., Cambridge, MA (US)

(72) Inventors: Jeffrey Peisner, Durham, NC (US); Andrew Harlan, Somerville, MA (US); Samuel Kesner, Arlington, MA (US); Gene Tacy, Windham, NH (US); Christopher Long, Willoughby, OH (US); Justin LaRue, Somerville, MA (US)

(21) Appl. No.: 17/157,154

(22) Filed: Jan. 25, 2021

Related U.S. Application Data
(60) Provisional application No. 62/994,529, filed on Mar. 25, 2020.

(51) Int. Cl. A61F 5/01; B25J 9/00

(52) U.S. Cl. CPC A61F 5/013; A61F 2005/0144; B25J 9/0006

(57) ABSTRACT

A compact, powered, orthotic device for pediatric use enables the user to control relative motion of the upper arm and the forearm about the elbow and grasping motions of the thumb and fingers. The device is powered by a set of battery-driven, backdrivable linear actuators that are positioned remotely from an arm of the subject. Control of motion of the device by the subject occurs by means of electromyographic signals from a sensor array in contact with skin on the arm of the subject. The sensors in the sensor array may be held in place on the forearm, on the upper arm, or at any other convenient location on the arm.

### CROSS-REFERENCE TO RELATED APPLICATIONS

This patent application claims the benefit of U.S. Provisional Patent Application No. 62/994,529, filed Mar. 25, 2020, the disclosure of which is incorporated by reference herein in its entirety.

### GOVERNMENT SUPPORT

Government funding for this invention was provided by Grant No. 4P50FD004911-04; FAIN P50FD004911 (CFDA number 93.103), awarded to the Children's Hospital of Philadelphia by the Food and Drug Administration. The government has certain rights in the invention.

### TECHNICAL FIELD

The present invention generally relates to powered orthotic devices, and more particularly to powered orthotic devices suitable as rehabilitation or functional aids for pediatric use.

### BACKGROUND ART

There are a number of neuromuscular and neurological conditions that prevent children from volitionally moving their arms and hands. Examples include cerebral palsy, brachial plexus injuries, traumatic spinal cord or brain injuries, stroke, and genetic and disease-related paralyses such as muscular dystrophy.

Currently the main options for these children are rigid splints, physical therapy, medications to treat muscle tightness and spasticity, and external electrical stimulation of the muscles. In some cases surgery may be attempted in order to transfer nerves or muscles and thereby regain some function of the impaired limb.

### SUMMARY OF THE EMBODIMENTS

Embodiments of the invention provide a powered orthotic device for use in assisting in relative motion of body parts of a subject, the body parts being selected from the group consisting of (a) a forearm and an upper arm, (b) a thumb and a set of fingers, and (c) combinations thereof. In this embodiment, the device includes:

(A) at least one assembly selected from the group consisting of
(1) an arm assembly, having
   (a) an upper arm module, an upper arm cuff coupled to the upper arm module, configured to removably attach the upper arm module to an upper arm of the subject,
   (b) a forearm module, a forearm cuff coupled to the forearm module, configured to removably attach the forearm module to a forearm of a subject,
   (c) an elbow module movably linking the upper arm module and the forearm module,
(2) a hand assembly, having a grasp module including a finger saddle that is removably attachable to a set of fingers and a thumb saddle that is removably attachable to a thumb, and
(3) combinations of the arm assembly and the hand assembly;
   (B) a set of electrically powered, backdrivable linear actuators, positioned remotely from an arm of the subject and configured to be coupled to the arm assembly via a set of arm cables and configured to be coupled to the hand assembly via a set of hand cables.

In this embodiment, the set of linear actuators is configured in relation to the arm assembly to cause relative motion of the upper arm module and the first forearm module, and therefore of the upper arm and the forearm, and the set of linear actuators is configured in relation to the hand assembly to cause relative motion of the finger saddle and the thumb saddle and therefore of the set of fingers and the thumb.

In a further related embodiment, wherein the at least one assembly includes the arm assembly, the set of arm cables includes a single elbow cable having a first end and a second end, and the set of linear actuators includes a first linear actuator, the elbow cable being attached at both ends to the first linear actuator, and routed to the elbow module over a set of pulleys, configured so as to cause relative motion of the upper arm module and the forearm module about an axis proximate to an elbow of the subject.

In a further related embodiment, wherein the at least one assembly includes the hand assembly, the set of hand cables includes a single hand cable having a first end and a second end, and the set of linear actuators comprising a second linear actuator, the hand cable being attached at both ends to the second linear actuator, and routed over an actuator pulley and a grasp pulley, configured so as to move the finger saddle and the thumb saddle together in a grasping motion as the actuator pulls the single hand cable in one linear direction, and to move the finger saddle and the thumb saddle apart in an ungrasping motion as the actuator pulls the single hand cable in an opposite linear direction.

In another related embodiment, wherein the at least one assembly includes the hand assembly, the set of hand cables includes a grasp cable having a first end and a second end, and the set of linear actuators comprising a second linear actuator, the grasp cable being attached at the first end to the second linear actuator and at the second end to a grasp attachment point on the grasp module, configured such that:
(i) when the second linear actuator causes motion of the first end of the grasp cable in a first linear direction, the grasp cable increases tension on the grasp attachment point, by pulling on the second end of the grasp cable, thereby causing the finger saddle to move towards the thumb saddle; and
(ii) when the second linear actuator causes motion of the first end of the grasp cable in a second linear direction, opposite the first linear direction, the grasp cable decreases tension on the grasp attachment point, by decreasing tension on the second end of the grasp cable, thereby causing the finger saddle to move away from the thumb saddle.

In another related embodiment, wherein the at least one assembly includes the arm assembly and the hand assembly, the hand assembly is coupled to the forearm module of the arm assembly.

In another related embodiment, the device further includes an electromyographic sensor array making electrical contact with skin of the arm, the sensor array being in electronic communication with the set of electrically powered actuators; wherein the set of electrically powered actuators is configured to respond to volitional electromyographic (EMG) signals from the EMG sensor array.

In a further related embodiment, the EMG sensor array is chosen from the group consisting of a set of upper arm EMG sensors, located on the upper arm of the subject, a set of forearm sensors, located on the forearm of the subject, and combinations thereof. Optionally, wherein the at least one assembly includes the arm assembly, the set of electrically powered actuators is configured to respond to volitional EMG signals from the EMG sensor array to cause relative motion of the upper arm and forearm modules of the arm assembly. Alternatively, or in addition, wherein the at least one assembly includes the hand assembly, the set of electrically powered actuators is configured to respond to volitional EMG signals from the EMG sensor array to move the grasp cable to cause relative motion of the finger saddle and the thumb saddle. Alternatively, or in addition, wherein the at least one assembly includes both the arm assembly and the hand assembly, the set of electrically powered actuators is configured to respond to volitional EMG signals from the EMG sensor array to cause relative motion of the upper arm and forearm modules of the arm assembly, and the set of electrically powered actuators is configured to respond to volitional EMG signals from the EMG sensor array to move the grasp cable to cause relative motion of the finger saddle and the thumb saddle. Optionally, the at least one assembly includes the upper-arm cuff, the upper arm cuff including a first hard shell attached to a first strap for removably securing the upper-arm cuff to the upper-arm. Optionally, the at least one assembly includes the forearm cuff, which includes a second hard shell attached to a second strap for removably securing the forearm cuff to the forearm.

In a further related embodiment, the first hard shell, further includes a first flexible sensor strap attached to the first hard shell, for situating the set of upper arm EMG sensors on the upper-arm, and the second hard shell, further includes a second flexible sensor strap attached to the second hard shell, for situating the set of forearm EMG sensors on the forearm.

In a further related embodiment, the grasp module further includes a third hard shell attached to a third strap for removably securing the grasp module to the hand.

In a further related embodiment, a module chosen from the group consisting of the upper arm module, the forearm module, and combinations thereof, have length adjustments to accommodate a range of upper arm and forearm sizes.

In a further related embodiment, the electrically powered, backdrivable set of linear actuators includes at least one ball screw actuator.

### BRIEF DESCRIPTION OF THE DRAWINGS

The foregoing features of embodiments will be more readily understood by reference to the following detailed description, taken with reference to the accompanying drawings, in which:

FIG. 1A is a perspective view of a first embodiment of a powered orthotic device in accordance with the present invention, shown as attached to an arm. In this embodiment, the cables are hidden from view within a cable sheath and a protective casing.

FIG. 1B is a perspective view of a second embodiment of a powered orthotic device in accordance with the present invention shown as worn by a subject, wherein a set of linear actuators is carried in a backpack on the subject.

FIG. 1C is a perspective view of a third embodiment of a powered orthotic device worn on a patient, where the set of linear actuators is held in a sling on the subject.

FIG. 1D is a perspective view of a fourth embodiment of a powered orthotic device worn on a patient, where the set of linear actuators is held in a bag on a wheelchair on the subject. In this embodiment, the device has a forearm module and a hand assembly, but no arm assembly.

FIG. 2 is a perspective view of an embodiment of a powered orthotic device according to the instant invention including a set of linear actuators, an arm assembly including a forearm module, a forearm cuff attached to the forearm module, an upper arm module, an upper arm cuff attached to the upper arm module and an elbow module, a hand assembly including a grasp module and a rigid hand coupling and cables connecting the set of linear actuators with the elbow assembly and the grasp module. In this figure, the protective sheath and protective casing are not shown, in order to illustrate the disposition of the grasp cable and the two ends of the elbow cable.

FIG. 3A is a perspective view of a first embodiment of the present invention with a linear actuator assembly configured for moving an upper arm module with respect to a forearm module about an axis proximate to an elbow.

FIG. 3B is a perspective view of the linear actuator assembly of FIG. 3A with the cover removed, showing a ball screw mechanism with a translationally movable arm cable.

FIG. 4A is a perspective view of an embodiment of the present invention, showing an arm assembly with the cover removed. To the right of the elbow module in FIG. 4A is a forearm bracket, forming part of the forearm module. To the left of the elbow module is an upper arm bracket, forming part of the upper arm module.

FIG. 4B is a perspective view of a pulley mechanism within the arm assembly of FIG. 4A. In this embodiment, two pulleys are attached to the forearm module.

FIG. 4C is a side view of the fully extended arm assembly according to FIG. 4A.

FIG. 4D is a side view of the flexed arm assembly according to FIG. 4A.

FIG. 5A is a perspective view of a second embodiment of a linear actuator assembly configured both for flexion and extension of the forearm and upper arm about the elbow and for grasping of a thumb and a set of fingers.

FIG. 5B is an exploded view of the underside of the linear actuator assembly of FIG. 5A, allowing visualization of a first motor, a second motor, and a battery. For clarity, the cables are not shown in this view.

FIG. 5C is another exploded view of the linear actuator assembly of FIG. 5A, with the top cover removed, showing the actuator mechanism and how it connects to the first motor and the second motor. For clarity, the cables are not shown in this view.

FIG. 5D is a top-down view of the linear actuator assembly of FIG. 5A, with the casing removed and the grasp cables and the arm cables shown.

FIG. 6A is a top view of an embodiment of a spring-based grasp module, with cover removed, shown in an ungrasped position with the spring tension released and the thumb saddle separated from the finger saddle.

FIG. 6B is a top-down view of the embodiment of the spring-based grasp module of FIG. 6A, with cover removed, shown in a grasping position, with the spring extended and the thumb saddle moved towards the finger saddle.

FIG. 6C is a side view of the embodiment of the spring-based grasp module of FIG. 6A, shown in an ungrasped position, with the spring tension extended and the thumb saddle moved towards the finger saddle.

FIG. 6D is a side view of the embodiment of the spring-based grasp module of FIG. 6A, shown in a grasping position, with the spring tension released and the thumb saddle moved away from the finger saddle.

FIG. 7A is a perspective view of an embodiment of a pulley-based grasp module, gear cover attached, with the finger saddle bracket positioned so that the finger saddle and the thumb saddle are moved towards each other in a grasping motion.

FIG. 7B is a perspective view of the embodiment of the pulley-based grasp module of FIG. 7A, gear cover attached, with the finger saddle bracket positioned so that the finger saddle and the thumb saddle are moved away from each other in an ungrasping motion.

FIG. 7C is an interior view of the embodiment of the pulley-based grasp module of FIG. 7A, with the cover removed, showing the grasp module pulley and gear mechanism. In this view the finger saddle and the thumb saddle are moved towards each other in a grasping motion.

FIG. 7D is an interior view of the embodiment of the pulley-based grasp module of FIG. 7A with the cover removed, showing the grasp module pulley and gear mechanism. In this view the finger saddle and the thumb saddle are moved away from each other in an ungrasping motion.

FIG. 7E is a back side view of the embodiment of the pulley-based grasp module of FIG. 7A, with a communications cable and a magnetic encoder shown.

FIG. 7F is an interior view of the gear cover of the embodiment of the pulley-based grasp module of FIG. 7A, that confines the grasping motion to a range of less than about 90 degrees, preferably less than about 70 degrees.

FIG. 8 is a cut-out view of a ball screw mechanism, as embodied in the actuator assemblies of FIGS. 3A-3B and 5A-5D, showing the manner in which the bearings are configured between nut and screw threads.

FIG. 9A is a side view of an embodiment of the present invention, providing an upper-arm cuff with structural straps and sensor straps.

FIG. 9B is a side view of the embodiment of the upper-arm cuff of FIG. 9A with the straps extended, showing the positioning of an array of EMG sensors.

### DETAILED DESCRIPTION OF SPECIFIC EMBODIMENTS

### Definitions

As used in this description and the accompanying claims, the following terms shall have the meanings indicated, unless the context otherwise requires:

A “linear actuator” is a motorized device that uses energy to produce linear motion.

A “set” includes at least one member.

A “set of linear actuators” is synonymous with “one or more linear actuators.”

An “actuator assembly” is synonymous with “a set of linear actuators.”

An “electrical linear actuator” translates electrical energy into linear mechanical motion. It may include an electrically powered motor that generates rotational motion, and a lead screw or ball screw assembly that converts the rotational motion to linear motion.

A “backdrivable linear actuator” is a low-friction, high efficiency linear actuator that, when power is turned off, will move easily in either linear direction upon application of a load.

To “translate” means to move in a linear manner, i.e. in one of two directions.

“Translational motion” is synonymous with linear motion.

A “lead screw linear actuator assembly” is a linear actuator assembly that makes use of a nut with helical threads that moves linearly in a one direction when a screw assembly with matching helical threads is rotated clockwise through the nut, and moves linearly in the opposite direction when the screw assembly is rotated counter-clockwise. Because of the significant frictional force opposing linear motion, the lead screw mechanism is not considered “backdrivable” and will maintain a significant load when power is turned off.

A “ball screw linear actuator assembly” is a linear actuator assembly that makes use of ball bearings that slide along circular threads on both a screw and a nut in order to convert rotational to translational motion. It functions in a similar manner to the lead screw linear actuator assembly in that the direction of linear motion of the nut depends on the direction of rotation of the screw. However, for the ball screw mechanism, the nut and screw thread do not make contact directly, but move along using ball bearings rolling between them. As a consequence, ball screw mechanisms have significantly reduced friction compared to lead screw mechanisms, allowing them to achieve 70% to 95% efficiency. As a further consequence, the ball screw mechanism is “backdrivable”.

An “actuator pulley” is a pulley disposed within an actuator assembly, around which a cable is routed to allow linear motion of an actuator to pull on the cable in either of two opposite linear directions.

A “grasp pulley” is a pulley disposed within a grasp module that allows linear motion of a cable to be transduced into grasping and ungrasping motions of a finger saddle with respect to a thumb saddle.

An “electromyographic sensor array” is an array of electromyographic sensors that in the context of this invention are situated on the arm of the subject, making contact with the skin on the arm.

An “upper arm electromyographic sensor array,” also called an “upper arm EMG sensor array,” is an array of electromyographic (EMG) sensors situated on the upper arm, the sensors being in electrical contact with skin on the upper arm.

A “forearm electromyographic sensor array,” also called a “forearm EMG sensor array” is an array of EMG sensors situated on the forearm, the sensors being in electrical contact with skin on the forearm.

By “relative motion” about an axis is meant at least one of flexion and extension motion about the axis.

“About” a numerical value means within ±10% of the numerical value.

Leveraging the technology we have successfully developed with adults with upper limb impairment, as shown for example, in US 20160287422, embodiments of the present invention will assist children with arm and hand function by using their own biofeedback signals (EMG, EEG, etc.) to control a compact, portable, motorized prosthesis, appropriate for pediatric use, having a set of one or more electrically powered, backdrivable linear actuators, positioned remotely from an arm of a subject, configured to aid children with flexion and extension movements about the elbow, and with grasping movements that bring together a set of fingers and a thumb. In specific embodiments, the set of linear actuators may be carried in a backpack or sling on the back of a patient, or hung on the back of a wheelchair.

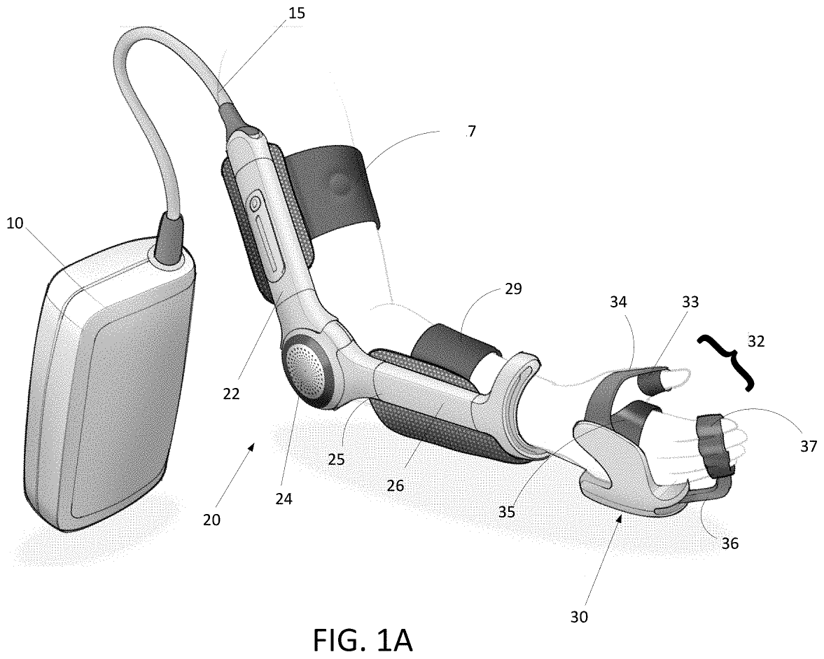

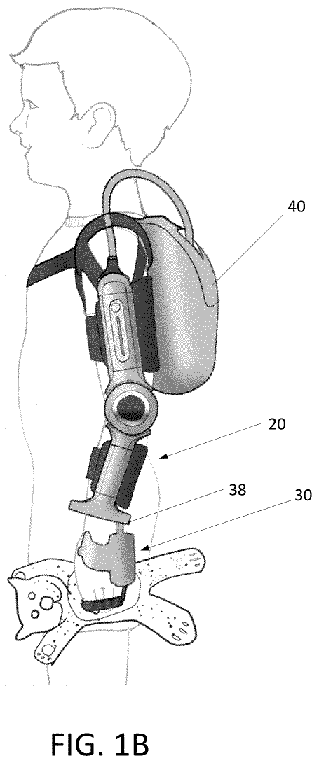

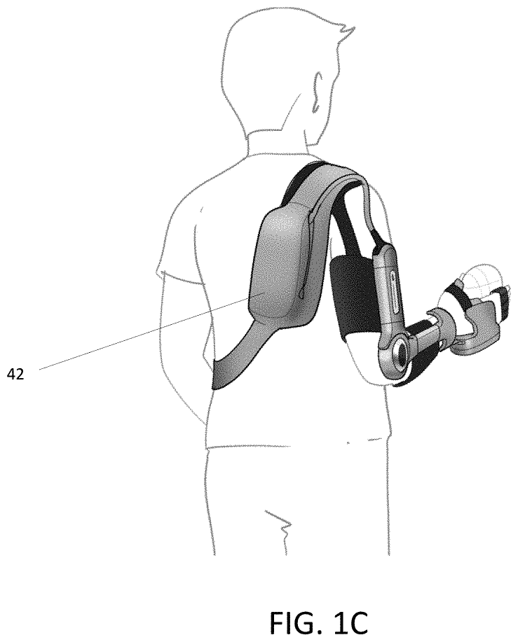

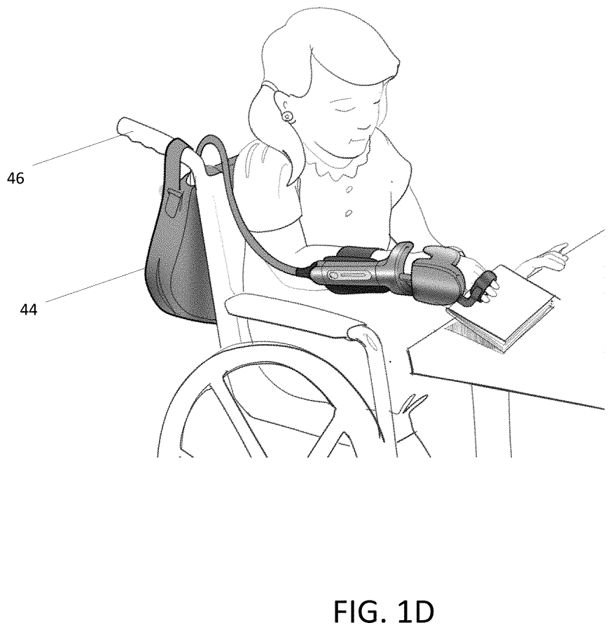

FIG. 1A is a perspective view of a first embodiment of a powered orthotic device in accordance with the present invention, shown as attached to an arm. In this embodiment, the cables are hidden from view within a cable sheath and a protective casing. In this embodiment, the powered orthotic device is configured for flexion/extension about the elbow and for grasping motions. The embodiment includes an actuator assembly 10 including a set of electrically powered, backdrivable, linear actuators, an arm assembly 20, and a hand assembly 30. The arm assembly 20 has an upper arm module 22, an elbow module 24, and a forearm module 26. The upper arm module 22 has an upper arm cuff 27 for securing the upper arm module 22 to the upper arm, and the forearm module 26 has a forearm cuff 29 for securing the forearm module 26 to the forearm. The elbow module 24 is configured for flexion and extension motions of the forearm with respect to the upper arm. The hand assembly 30 includes a grasp module 32. The grasp module 32 includes a thumb saddle 34 and a finger saddle 36. As shown in FIG. 1A, the thumb of a subject is attached to the thumb saddle 34 by means of a thumb brace 33 and the set of fingers is attached to the finger saddle 36 by means of a finger brace 37. Mechanical cables and communications cables from the set of linear actuators 10 are in this view encased in cable sheath 15 and are further hidden from view by a protective casing 25 extending from the upper arm assembly to the forearm module 26.

FIG. 1B is a perspective view of a second embodiment of a powered orthotic in accordance with the present invention, shown as worn by a subject, wherein the actuator assembly 10 is disposed within a backpack 40 worn on the subject. Also shown in this figure is a rigid hand coupling 38 that connects the forearm module 26 and the hand assembly 30.

FIG. 1C is a perspective view of a third embodiment of a powered orthotic device worn on a patient, where the set of linear actuators 10 is held in a sling 42 on the subject.

FIG. 1D is a perspective view of a fourth embodiment of a powered orthotic device worn on a subject, where the set of linear actuators 10 is held in a bag 44 on the back of a wheelchair 46. In this embodiment, the device has a forearm module 26 and a hand assembly 30, but no arm assembly.

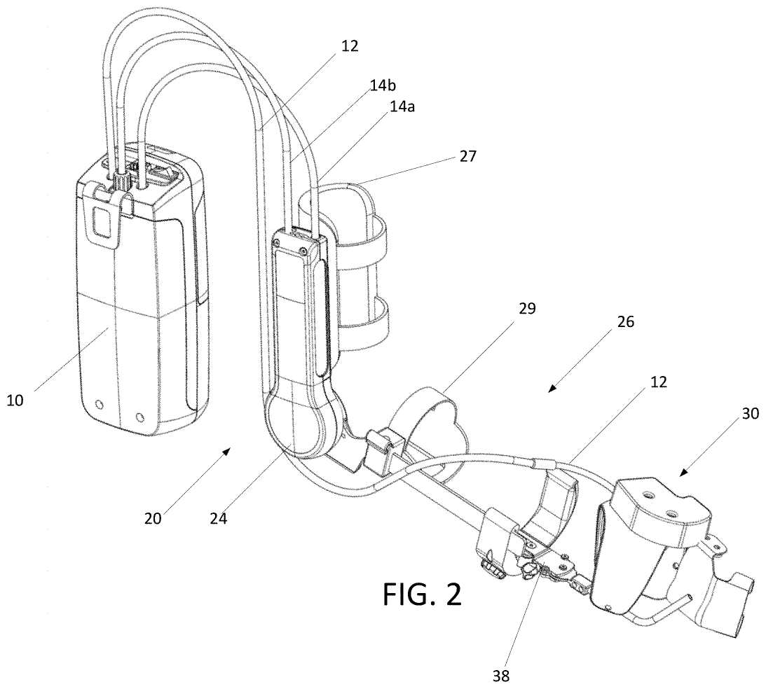

FIG. 2 is a perspective view of an embodiment of a powered orthotic device according to the instant invention including a set of linear actuators, an arm assembly 20 including a forearm module 26, a forearm cuff 29 attached to the forearm module 26, an upper arm module 22, an upper arm cuff 27 attached to the upper arm module 22 and an elbow module 24, a hand assembly 30 including a grasp module 32 and a rigid hand coupling 38 and cables connecting the set of linear actuators 10 with the elbow module 24 and the grasp module 32. In this figure, the protective sheath 15 and protective casing 25 are not shown, in order to illustrate the disposition of the grasp cable 12 and the two ends of the elbow cable 14a and 14b.

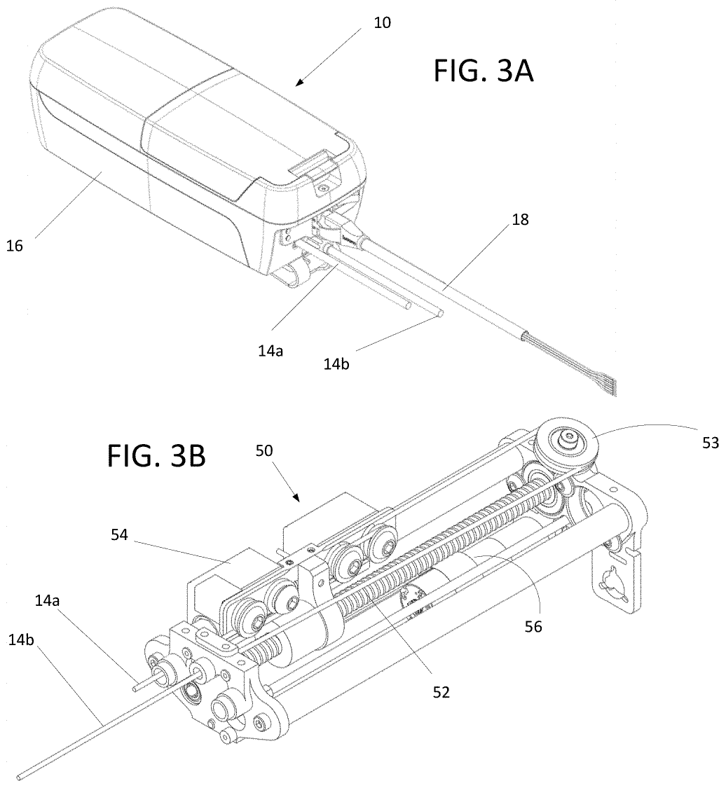

FIG. 3A is a perspective view of a first embodiment of a linear actuator assembly configured for moving an upper arm module 22 with respect to a forearm module 26 about an axis proximate to an elbow. In this embodiment, the set of linear actuators 10 has a first linear actuator 50 configured with an elbow cable with two ends, 14a and 14b, for moving the upper arm module 22 with respect to the forearm module 26 about an axis proximate to an elbow. Shown in this view are the two ends of the elbow cable 14a, 14b as they enter the actuator assembly 10, and an electrical cable 18 for conveying electromyographic signal information to control the linear motion of the two ends of the elbow cable 14a and 14b. The electrical cable 18 is configured to relay signals based on EMG sensors making contact with skin on the arm in order to control the motion of the first linear actuator 50.

FIG. 3B is a perspective view of the linear actuator assembly of FIG. 3A with the cover removed, showing a ball screw mechanism. Both ends of the elbow cable 14a and 14b are attached to the same elbow cable carriage 54. The elbow cable carriage 54 is disposed on an elbow cable track 52 of the first linear actuator assembly 50. A first electric motor 56 is configured as described below to cause rotation of the elbow cable track in either a clockwise or a counter-clockwise direction. As the elbow cable track 52 rotates in a clockwise direction, the elbow cable carriage 54 moves in one linear direction along the elbow cable track 52, whereas when the rotor turns counter-clockwise, the elbow cable carriage 54 moves in the other linear direction along the elbow cable track 52. Motion of the carriage 54 in one or the other direction causes the elbow cable to move about the rotating actuator pulley 53, in a clockwise, or counterclockwise direction.

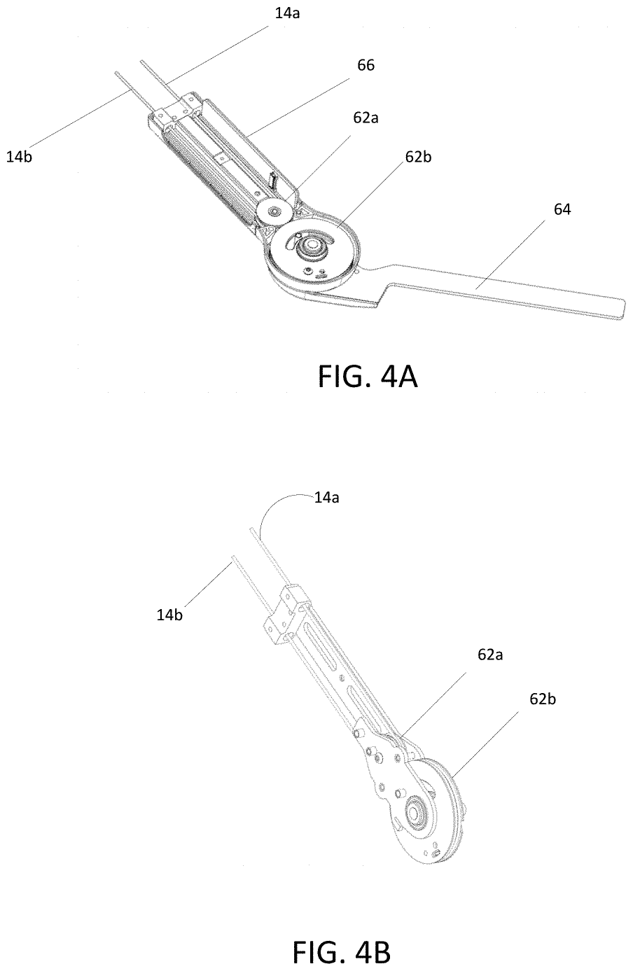

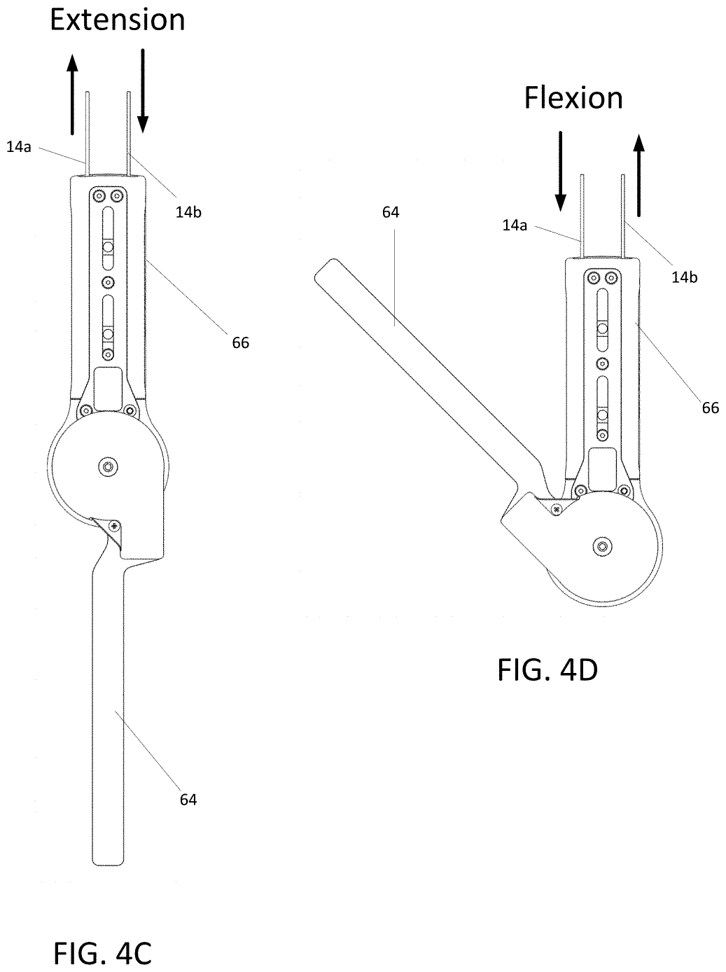

FIG. 4A is a perspective view of an embodiment of an arm assembly with the cover removed. To the right of the elbow module 24 in FIG. 4A is a forearm bracket 64, forming part of the forearm module 26. To the left of the elbow module 24 is an upper arm bracket 66, forming part of the upper arm module 22.

FIG. 4B is a perspective view of the pulley mechanism for the embodiment of FIG. 4A. In this embodiment, two pulleys, 62a and 62b, are attached to the forearm module 26.

FIG. 4C is a side view of the fully extended arm assembly in the embodiment of FIG. 4A, and FIG. 4D is a side view of the flexed arm assembly in the embodiment of FIG. 4A. As is illustrated in FIGS. 4C and 4D, pulling on the 14a cable end results in extension about the elbow, whereas pulling on the 14b cable end results in flexion.

In the embodiment of FIGS. 4A-4D, movement of the elbow cable carriage 54 in one direction is transduced into flexion of the forearm module 26 with respect to the upper arm module 22 about the elbow module 24 by means of a set of one or more pulleys 62 in the elbow module. Movement of the elbow cable carriage 54 in the other direction is transduced into extension of the forearm module 26 with respect to the upper arm module 22 about the elbow module 24 by means of the set of one or more pulleys 62 in the elbow module.

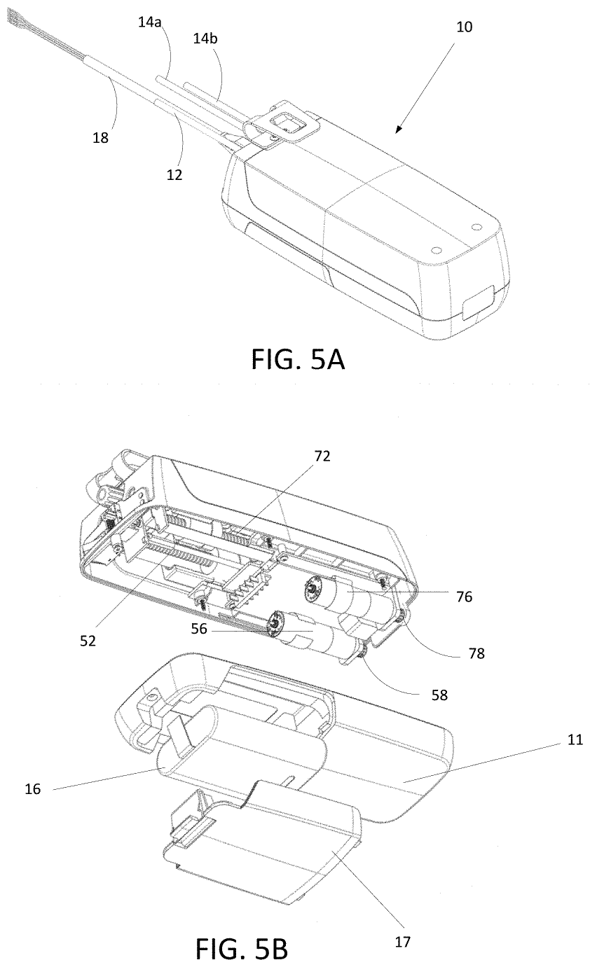

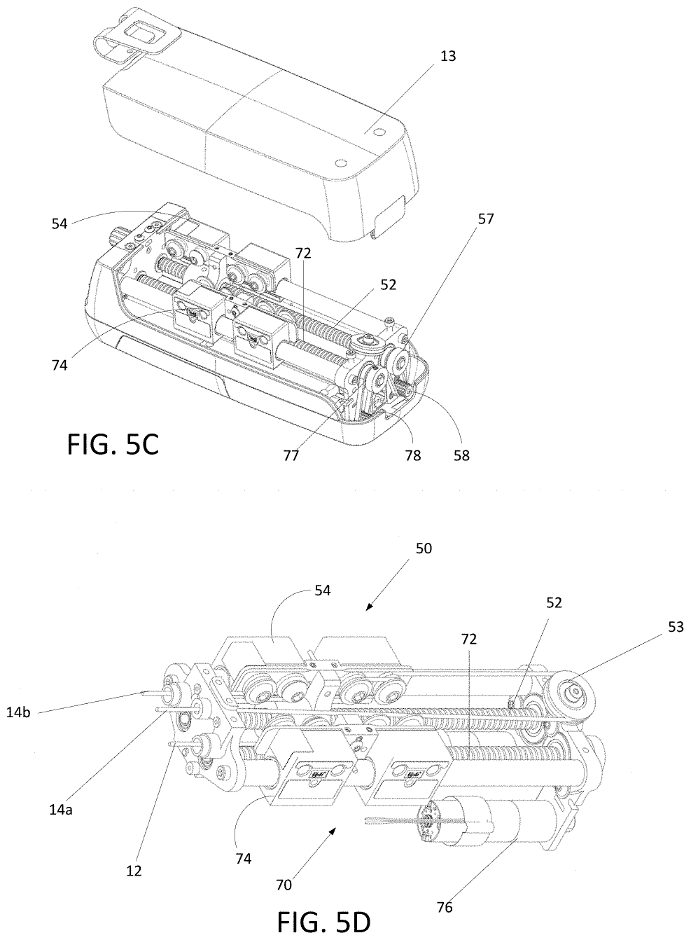

FIGS. 5A-5D show a second embodiment of a linear actuator assembly 10. In this embodiment, the linear actuator assembly is attached to an elbow cable with two ends 14a and 14b for moving the upper arm module 22 with respect to the forearm module 26 about an axis proximate to an elbow, and to a grasp cable 12 for moving the thumb saddle with respect to the finger saddle.

In the embodiment of FIGS. 5A-5D the elbow cable ends 14a and 14b are both attached to the elbow cable carriage 54 of the first linear actuator 50 and can move linearly in one direction or the other, as described above, to cause extension or flexion of the upper arm and the forearm about the elbow. In this embodiment, one end of the grasp cable 12 is attached to a grasp cable carriage 74 of a second linear actuator 70 and can move linearly in one direction or the other as the grasp cable moves, to cause relative motion of the thumb saddle with respect to the finger saddle.

FIG. 5A is a perspective view of this second embodiment of a linear actuator assembly, configured both for flexion and extension of the forearm and upper arm about the elbow and for grasping of a thumb and a set of fingers. Shown in FIG. 5A are the two ends of the elbow cable, 14a and 14b as they enter the actuator assembly 10, one end of the grasp cable 12 as it enters the actuator assembly 10 and the electrical cable 18 for conveying EMG signal information to control the linear motions of the elbow and grasp cables. The electrical cable connects to EMG sensors on the upper arm and forearm modules, the sensors making contact with skin on the upper arm and forearm.

FIG. 5B is an exploded view of the underside of the actuator assembly 10 of FIG. 5A with the bottom cover 11 removed, allowing visualization of a first motor, a second motor, and a battery. For clarity, the cables are not shown in this view. The first motor 56 drives the first linear actuator, and a second motor 76 drives the second linear actuator. A battery 16 fits into the bottom cover, and is covered by a battery cover 17. The battery provides electrical power to drive the first motor 56 and the second motor 76. Electrical power supplied by the battery causes the first motor rotor 58 and the second motor rotor 78 to rotate in clockwise or counter-clockwise directions.

FIG. 5C is another exploded view of the linear actuator assembly 10 of FIG. 5A, with the top cover 13 removed, showing the actuator mechanism and how it connects to the first motor 56 and the second motor 76. In this figure as well, the cables are not shown. Here, it is seen that first motor rotor 58 is connected by a first belt 57 that goes around the end of the elbow track 52. As the first motor rotor 58 rotates, this causes the first belt 57 to turn, which causes the elbow track 52 to rotate in the same direction as the first motor rotor 58. Similarly, as the second motor rotor 78 rotates, this causes the grasp cable track 72 to rotate in the same direction as the second motor rotor 78.

FIG. 5D is a top-down view of the linear actuator assembly 10 of FIG. 5A, with the casing removed and the grasp cable 12 and the arm cable 14a, 14b shown. FIG. 5D shows how the elbow cable ends 14a and 14b are connected to the elbow cable carriage 54, and how the grasp cable 12 is attached at one end to the grasp cable carriage 74.

In the embodiment of FIGS. 5A-5D, clockwise rotation of the first motor rotor 58 causes clockwise rotation of the elbow cable track 52, in turn causing the elbow cable carriage 54 to move in one linear direction along the elbow cable track 52, whereas when the first motor rotor turns counter-clockwise, the elbow cable carriage 54 moves in the other linear direction along the elbow cable track 52. Motion of the carriage in one or the other direction causes the elbow cable to move about the rotating actuator pulley 53, in a clockwise, or counterclockwise direction.

Similarly, rotation of the second motor rotor 78 in one direction causes the grasp cable carriage 74 to move in one linear direction along the grasp cable track 72 to pull on the grasp cable 12. Rotation of the second motor rotor in the other direction causes the grasp cable carriage 72 to move in the other linear direction to release tension on the grasp cable 12.

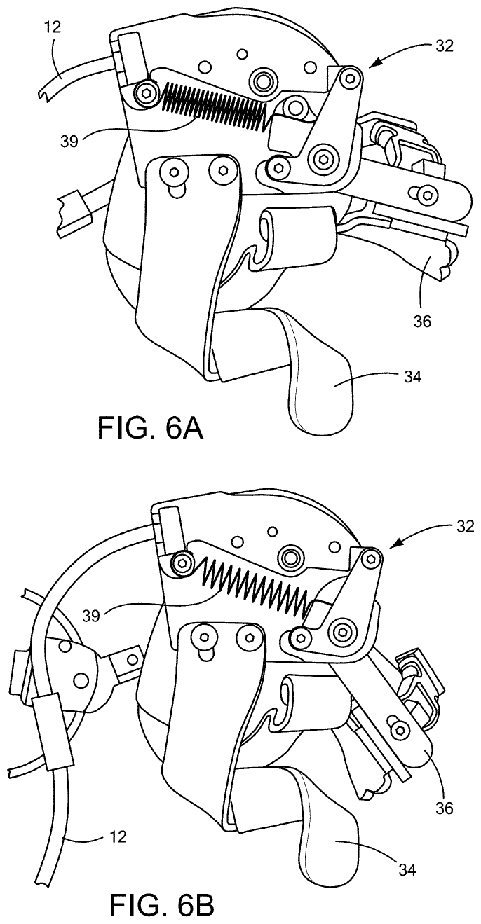

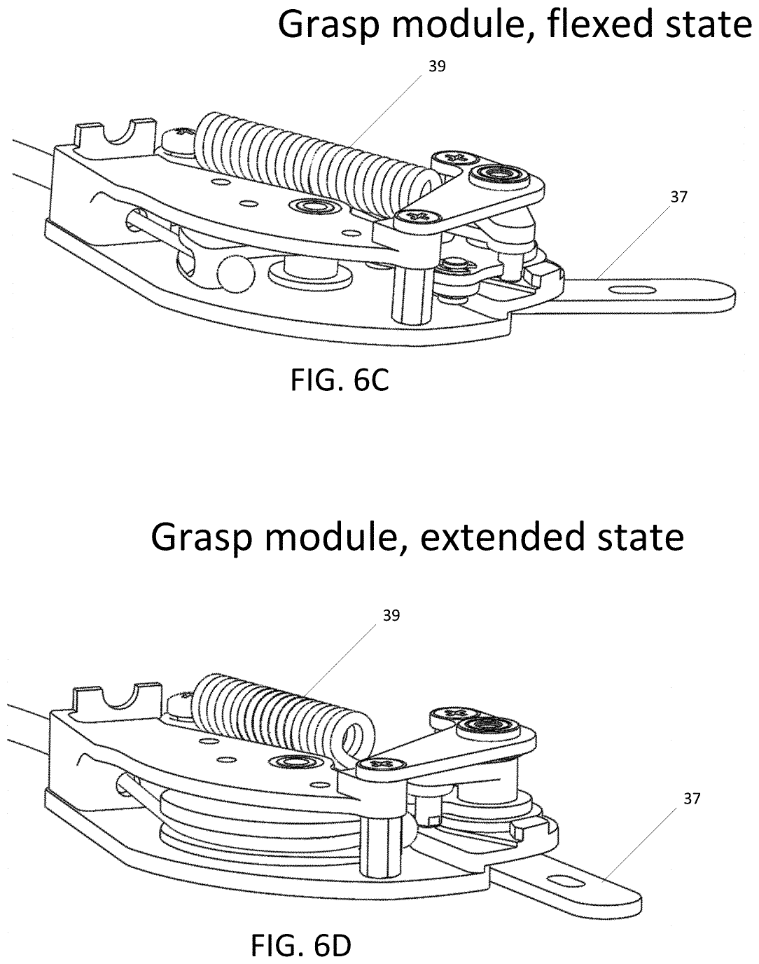

In the grasp module embodied in FIGS. 6A-6D, the grasp module 32 operates by a spring mechanism.

FIG. 6A is a top view of this embodiment, with cover removed, shown in an ungrasped position with the spring tension released and the thumb saddle 34 separated from the finger saddle 36.

FIG. 6B is a top-down view of the embodiment of the spring-based grasp module of FIG. 6A, with cover removed, shown in a grasping position, with the spring 39 extended and the thumb saddle 34 moved towards the finger saddle 36.

FIG. 6C is a side view of the embodiment of the spring-based grasp module 32 of FIG. 6A, shown in an ungrasped position, with the spring tension extended and the thumb saddle 34 moved towards the finger saddle 36.

FIG. 6D is a side view of the embodiment of the spring-based grasp module of FIG. 6A, shown in a grasping position, with the spring tension released and the thumb saddle 34 moved away from the finger saddle 36.

In this embodiment, one end of the grasp cable 12 is attached to a linear actuator 70, and the other end is attached to the grasp module 32. When the grasp cable carriage 74 moves in one linear direction, the grasp cable 12 is pulled, and the spring 39 extends, causing the finger saddle 36 and the thumb saddle 34 to move together in a grasping manner. When the grasp cable carriage 74 moves in the other linear direction, tension is released on the grasp cable 12, and the spring 39 contracts, causing the finger saddle 36 and the thumb saddle 34 to move apart, releasing the grasp.

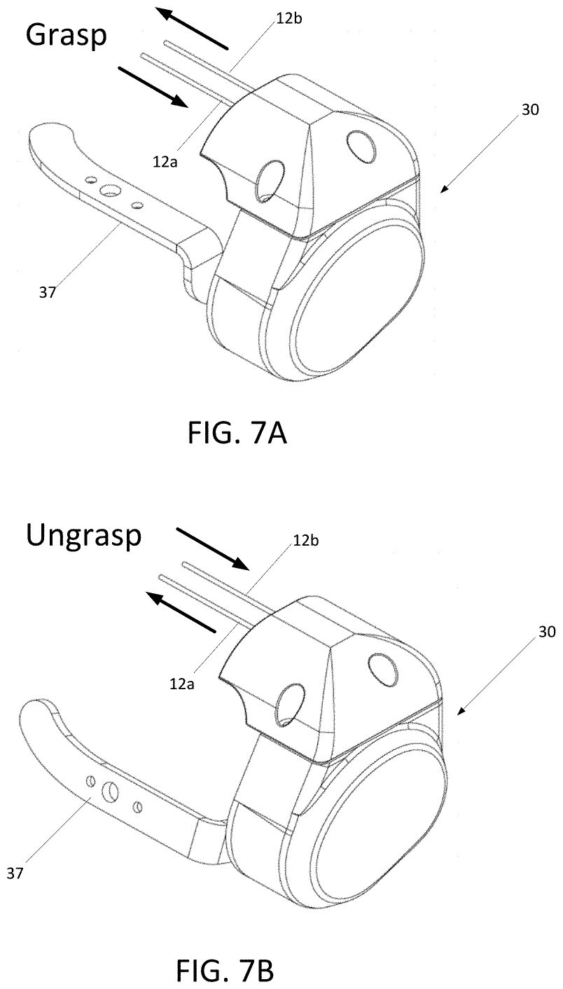

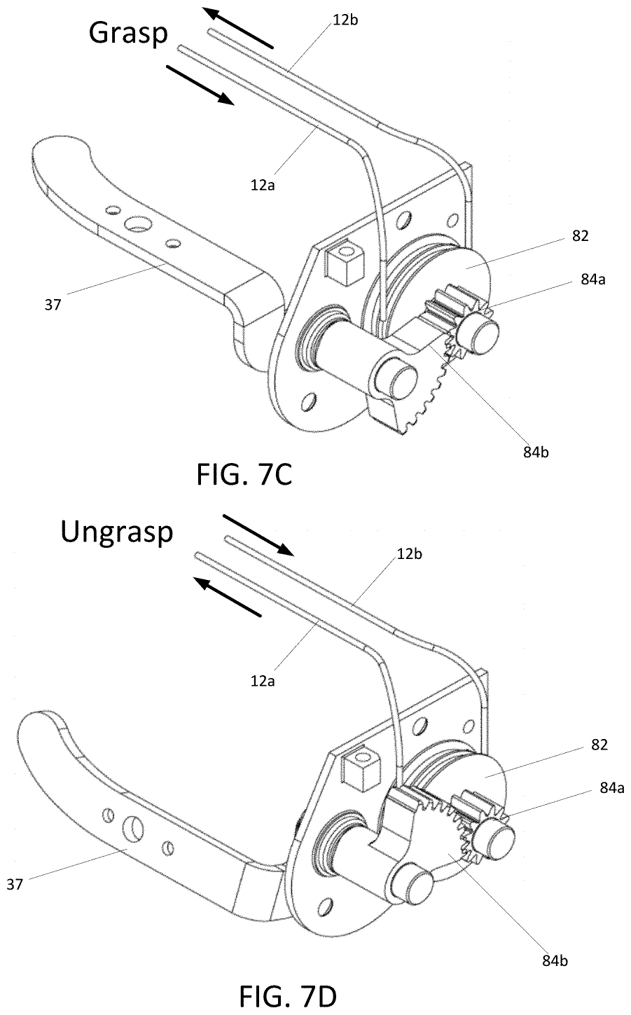

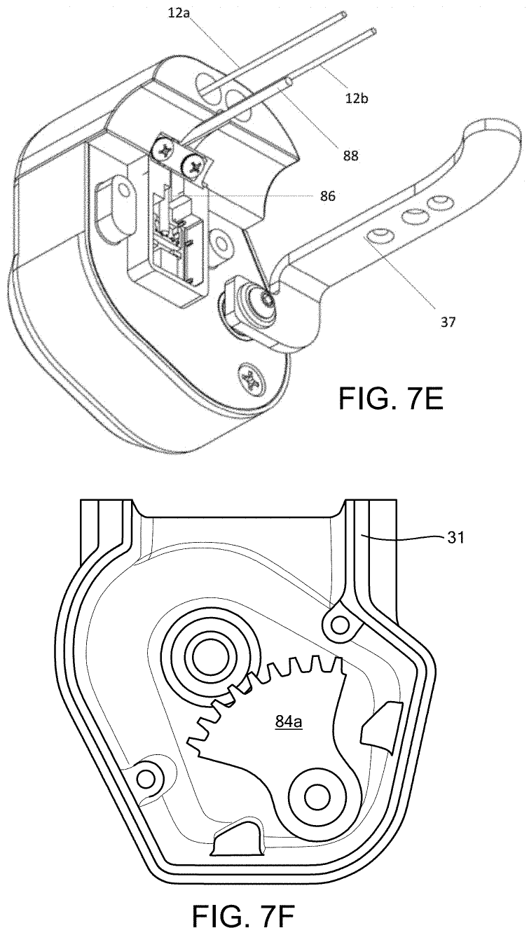

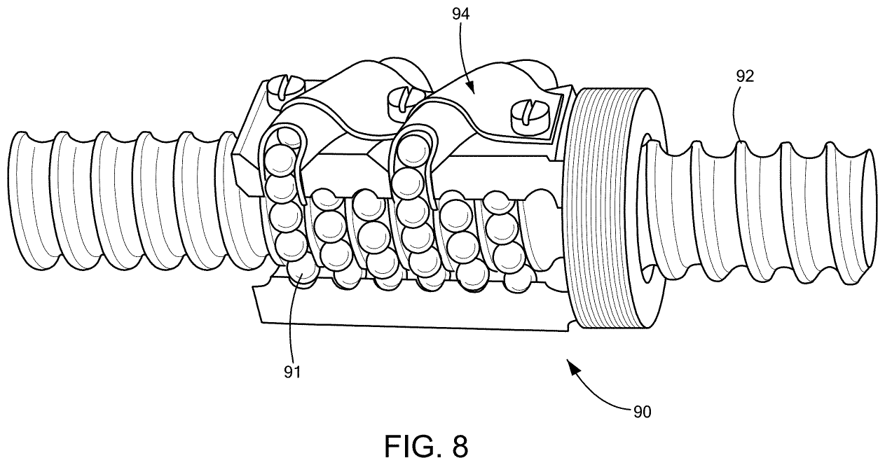

In the grasp module 30 embodied in FIGS. 7A-7F, the grasp module 30 operates by a pulley mechanism analogous to the elbow module 24.

FIG. 7A is a perspective view of the embodiment, gear cover attached, with the finger saddle bracket positioned so that the finger saddle 36 and the thumb saddle 34 are moved towards each other in a grasping motion.

FIG. 7B is a perspective view of the embodiment of the pulley-based grasp module of FIG. 7A, gear cover attached, with the finger saddle bracket positioned so that the finger saddle 36 and the thumb saddle 34 are moved away from each other in an ungrasping motion.

FIG. 7C is an interior view of the embodiment of the pulley-based grasp module of FIG. 7A, with the cover removed, showing the grasp module pulley 82 and gear mechanism 84a and 84b. In this view the finger saddle 36 and the thumb saddle 34 are moved towards each other in a grasping motion.

FIG. 7D is an interior view of the embodiment of the pulley-based grasp module of FIG. 7A with the cover removed, showing the grasp module pulley 82 and gear mechanism 84a and 84b. In this view the finger saddle 36 and the thumb saddle 34 are moved away from each other in an ungrasping motion.

In this embodiment, a single grasp cable is attached at both ends, 12a and 12b, to the second linear actuator and, as shown in FIGS. 7C and 7D, is routed to the grasp module 30 over an actuator pulley to a grasp module pulley 82. The grasp module pulley 82 is rotatably attached a first gear 84a, which is further connected to a second gear 84b, configured to transduce linear motion of the linear actuator in one linear direction into a grasping motion and to transduce linear motion in the opposite linear direction into an ungrasping motion, by moving the finger saddle 36 relative to the thumb saddle 34.

FIG. 7E is a back side view of the embodiment of FIGS. 7A-7D, with a communications cable 88 and a magnetic encoder 86 shown. The angular extent of the grasping and ungrasping motion is monitored by the magnetic encoder 86, whereas the communications cable 88 provides a means for communicating the angular motion monitored by the magnetic encoder 86 back to the actuator assembly.

FIG. 7F is an interior view of the gear cover 31 of the pulley-based grasp module of FIG. 7A. The grasping motion is limited to a range of less than about 90 degrees by confining the second gear 84b within a gear cover 31, as shown in FIG. 7F. In a preferred embodiment the grasping motion is limited to a range of less than about 90 degrees, preferably less than about 70 degrees.

A preferred embodiment of a backdrivable linear actuator according to the present invention is a ball screw linear actuator 90. FIG. 8 is a cutout view of an embodiment of a backdrivable linear actuator 90 based on a ball screw mechanism, as used in the actuator assemblies of FIGS. 3A-3B and 5A-5D. In this mechanism, friction is reduced by interposing ball bearings between helical grooves on the carriage 94 and helical grooves on the screw track 92. As the screw track 92 turns clockwise, the carriage 94 moves linearly to the right. As the screw track turns counterclockwise, the carriage 94 moves linearly to the left. As the ball-bearings move toward the end of the track, a return channel (not shown) returns them to the front of the track, ensuring that bearings are always interposed between the carriage and the screw track.

In embodiments of this invention, arrays of EMG sensors are held in place at various locations on the skin of the arm. Such sensors can be held strapped into place under the forearm cuff 29, and/or under the upper arm cuff 27.

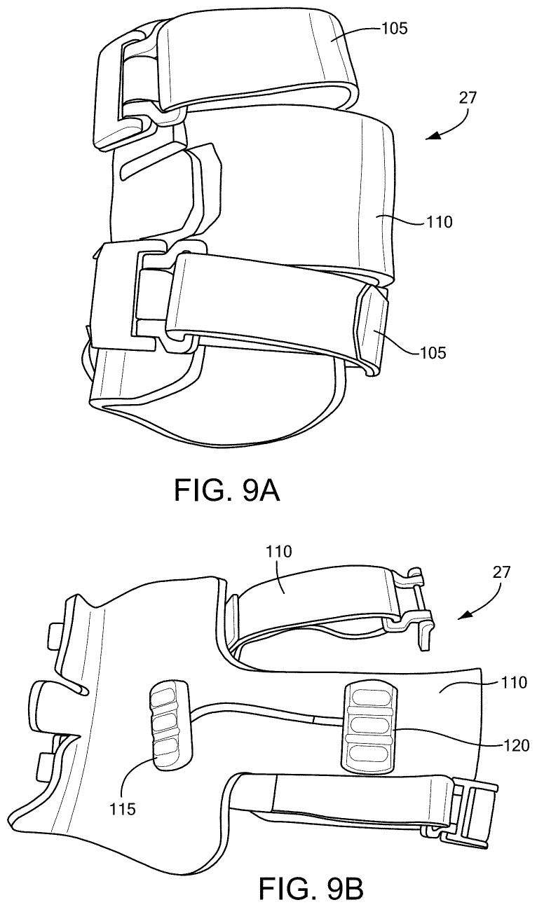

FIG. 9A is a side view of an embodiment of the present invention, providing an upper-arm cuff with structural straps 105 and sensor straps 110.

FIG. 9B is a side view of the embodiment of the upper-arm cuff of FIG. 9A with the straps extended, showing the positioning of an array of EMG sensors 120.

The upper arm cuff 27 in the embodiment of FIGS. 9A and 9B is held in place by structural straps 105 at the top and the bottom of the cuff. In between the structural straps is a sensor strap 110 having an array of sensors that includes a triceps sensor 115 and a biceps sensor 120. Having a sensor strap 110 that is separate from the structural straps 105 allows greater control of the amount of pressure that the sensors place on the skin of the arm, resulting in a more comfortable fit. Similarly, sensors can be placed on a sensor strap on the forearm cuff that is separately located from structural straps on the forearm cuff.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A powered orthotic device for use in assisting in relative motion of body parts of a subject, the body parts being selected from the group consisting of (a) a forearm and an upper arm, (b) a thumb and a set of fingers, and (c) combinations thereof, the device comprising:
(A) at least one assembly selected from the group consisting of
(1) an arm assembly, having
(a) an upper arm module, an upper arm cuff coupled to the upper arm module, configured to removably attach the upper arm module to an upper arm of the subject,
(b) a forearm module, a forearm cuff coupled to the forearm module, configured to removably attach the forearm module to a forearm of a subject, and
(c) an elbow module movably linking the upper arm module and the forearm module,
(2) a hand assembly, having a grasp module including a finger saddle that is removably attachable to a set of fingers and a thumb saddle that is removably attachable to a thumb, and
(3) combinations of the arm assembly and the hand assembly; and
(B) a set of electrically powered, backdrivable linear actuators, positioned remotely from an arm of the subject and configured to be coupled to the arm assembly via a set of arm cables and configured to be coupled to the hand assembly via a set of hand cables,
wherein the set of linear actuators is configured in relation to the arm assembly to cause relative motion of the upper arm module and the first forearm module, and therefore of the upper arm and the forearm, and wherein the set of linear actuators is configured in relation to the hand assembly to cause relative motion of the finger saddle and the thumb saddle and therefore of the set of fingers and the thumb.

2. The powered orthotic device according to claim 1, wherein the at least one assembly includes the arm assembly, the set of arm cables comprising a single elbow cable having a first end and a second end, and the set of linear actuators comprising a first linear actuator, the elbow cable being attached at both ends to the first linear actuator, and routed to the elbow module over a set of pulleys, configured so as to cause relative motion of the upper arm module and the forearm module about an axis proximate to an elbow of the subject.

3. The powered orthotic device according to claim 1, wherein the at least one assembly includes the hand assembly, the set of hand cables comprising a single hand cable having a first end and a second end, and the set of linear actuators comprising a second linear actuator, the hand cable being attached at both ends to the second linear actuator, and routed over an actuator pulley and a grasp pulley, configured so as to move the finger saddle and the thumb saddle together in a grasping motion as the actuator pulls the single hand cable in one linear direction, and to move the finger saddle and the thumb saddle apart in an ungrasping motion as the actuator pulls the single hand cable in an opposite linear direction.

4. The powered orthotic device according to claim 1, wherein the at least one assembly includes the hand assembly, the set of hand cables comprising a grasp cable having a first end and a second end, and the set of linear actuators comprising a second linear actuator, the grasp cable being attached at the first end to the second linear actuator and at the second end to a grasp attachment point on the grasp module, configured such that:
(i) when the second linear actuator causes motion of the first end of the grasp cable in a first linear direction, the grasp cable increases tension on the grasp attachment point, by pulling on the second end of the grasp cable, thereby causing the finger saddle to move towards the thumb saddle; and
(ii) when the second linear actuator causes motion of the first end of the grasp cable in a second linear direction, opposite the first linear direction, the grasp cable decreases tension on the grasp attachment point, by decreasing tension on the second end of the grasp cable, thereby causing the finger saddle to move away from the thumb saddle.

5. The powered orthotic device according to claim 1, wherein the at least one assembly includes the arm assembly and the hand assembly, and the hand assembly is coupled to the forearm module of the arm assembly.

6. The powered orthotic device according to claim 1, further comprising an electromyographic sensor array making electrical contact with skin of the arm, the sensor array being in electronic communication with the set of electrically powered actuators;
wherein the set of electrically powered actuators is configured to respond to volitional electromyographic (EMG) signals from the EMG sensor array.

7. The powered orthotic device according to claim 6, wherein the EMG sensor array is chosen from the group consisting of a set of upper arm EMG sensors, located on the upper arm of the subject, a set of forearm sensors, located on the forearm of the subject, and combinations thereof.

8. The powered orthotic device according to claim 7, wherein the at least one assembly includes the arm assembly,
the set of electrically powered actuators is configured to respond to volitional EMG signals from the EMG sensor array to cause relative motion of the upper arm and forearm modules of the arm assembly.

9. The powered orthotic device according to claim 7, wherein the at least one assembly includes the hand assembly,
the set of electrically powered actuators is configured to respond to volitional EMG signals from the EMG sensor array to move the grasp cable to cause relative motion of the finger saddle and the thumb saddle.

10. The powered orthotic device according to claim 7, wherein the at least one assembly includes the arm assembly and the hand assembly,
the set of electrically powered actuators is configured to respond to volitional EMG signals from the EMG sensor array to cause relative motion of the upper arm and forearm modules of the arm assembly, and
the set of electrically powered actuators is configured to respond to volitional EMG signals from the EMG sensor array to move the grasp cable to cause relative motion of the finger saddle and the thumb saddle.

11. The powered orthotic device according to claim 7, wherein the at least one assembly includes an arm assembly and the upper-arm cuff comprises a first hard shell attached to a first strap for removably securing the upper-arm cuff to the upper-arm, and the forearm cuff comprises a second hard shell attached to a second strap for removably securing the forearm cuff to the forearm.

12. The powered orthotic device according to claim 11, wherein the first hard shell further comprises a first flexible sensor strap attached to the first hard shell, for situating the set of upper arm EMG sensors on the upper-arm, and
wherein the second hard shell further comprises a second flexible sensor strap attached to the second hard shell, for situating the set of forearm EMG sensors on the forearm.

13. The powered orthotic device according to claim 1, wherein the at least one assembly includes the hand assembly and the grasp module further comprises a third hard shell attached to a third strap for removably securing the hand assembly to the hand.

14. The powered orthotic device according to claim 1, wherein the at least one assembly includes the arm assembly and the upper arm module, the forearm module, or both the upper arm module and the forearm module have length adjustments to accommodate a range of upper arm and forearm sizes.

15. The powered orthotic device according to claim 1, wherein the electrically powered, backdrivable set of linear actuators comprises at least one ball screw actuator.
